(19) United States
(12) Patent Application Publication
(10) Pub. No.: US 2024/0273540 A1
(43) Pub. Date: Aug. 15, 2024

(54) INTELLIGENT FILTERING OF TRANSACTIONS OVER A NETWORK USING LEARNED RULES

(71) Applicant: Surescripts, LLC, Arlington, VA (US)

(72) Inventors: Andrew Foster Mellin, Saint Paul, MN (US); Todd Michael Anderson, Minneapolis, MN (US)

(21) Appl. No.: 18/486,705

(22) Filed: Oct. 13, 2023

Related U.S. Application Data
(60) Provisional application No. 63/484,356, filed on Feb. 10, 2023.

(51) Int. Cl. G06Q 20/40

(52) U.S. Cl. CPC G06Q 20/405

(57) ABSTRACT

Methods, systems, and apparatuses are described for filtering a transaction over a network, according to embodiments. In an example system, a transaction request is retrieved over a network. A model that is configured to predict a likelihood associated with automatic adjudication of transactions is accessed. Based at least on the transaction request and the model, a likelihood that the transaction request can be adjudicated automatically is determined. If the likelihood that the transaction request can be adjudicated automatically is above a threshold, one or more actions are performed, such as generating an indication that a portion of the transaction request can be adjudicated automatically or generating an automatic adjudication indicating whether the transaction request should be approved. In response to generating the indication or the automatic adjudication, the transaction request and the indication or the automatic adjudication are forwarded over the network to an endpoint associated with the transaction request.

### CROSS-REFERENCE TO RELATED APPLICATION(S)

The present application claims priority to U.S. Provisional Patent Application No. 63/484,356, entitled “INTELLIGENT FILTERING OF TRANSACTIONS OVER A NETWORK USING LEARNED RULES,” and filed on Feb. 10, 2023, the entirety of which is incorporated by reference herein.

### BACKGROUND

### I. Technical Field

The present subject matter relates to techniques for filtering transactions over a network based on learned rules.

### II. Background Art

Electronic prior authorizations are authorization requests (e.g., for a medication) that are sent electronically over a network from one entity to another. In conventional systems, an electronic prior authorization requires answering a set of questions, which are filled in manually. The process typically requires an entity (e.g., a provider) to access an electronic health record and/or other sources of clinical information over a network to fill out answers to the questions, from which a determination can be made whether the authorization should be approved. Where billions of prescriptions need to be evaluated every year for a potential prior authorization, inefficiencies can result based on the systems and/or networks used for completing, reviewing, and/or approving the authorizations.

### BRIEF SUMMARY

Methods, systems, and apparatuses are described for filtering a transaction over a network based on learned rules, substantially as shown in and/or described herein in connection with at least one of the figures, as set forth more completely in the claims.

### BRIEF DESCRIPTION OF THE DRAWINGS/FIGURES

The accompanying drawings, which are incorporated herein and form a part of the specification, illustrate embodiments and, together with the description, further serve to explain the principles of the embodiments and to enable a person skilled in the pertinent art to make and use the embodiments.

FIG. 1 shows a block diagram of a system for intelligent filtering of a transaction over a network, according to an example embodiment.

FIG. 2 shows a flowchart of a method for intelligent filtering of a transaction over a network, in accordance with an example embodiment.

FIG. 3 shows a block diagram of a transaction interception system, according to an example embodiment.

FIG. 4 shows flowchart for a first illustrative implementation of a transaction interception, in accordance with an example embodiment.

FIG. 5 shows a flowchart for a second illustrative implementation of a transaction interception, in accordance with an example embodiment.

FIG. 6 shows a block diagram of a processing device/system in which the techniques disclosed herein may be performed and the embodiments herein may be utilized.

Embodiments will now be described with reference to the accompanying drawings. In the drawings, like reference numbers indicate identical or functionally similar elements. Additionally, the left-most digit(s) of a reference number identifies the drawing in which the reference number first appears.

### DETAILED DESCRIPTION

### I. Introduction

The present specification discloses numerous example embodiments. The scope of the present patent application is not limited to the disclosed embodiments, but also encompasses combinations of the disclosed embodiments, as well as modifications to the disclosed embodiments.

References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The use of “or” herein may be interchangeable with the term “and/or” unless otherwise stated.

If the performance of an operation is described herein as being “based on” one or more factors, it is to be understood that the performance of the operation may be based solely on such factor(s) or may be based on such factor(s) along with one or more additional factors. Thus, as used herein, the term “based on” should be understood to be equivalent to the term “based at least on.”

Furthermore, it should be understood that spatial descriptions (e.g., “above,” “below,” “up,” “left,” “right,” “down,” “top,” “bottom,” “vertical,” “horizontal,” etc.) used herein are for purposes of illustration only, and that practical implementations of the structures described herein can be spatially arranged in any orientation or manner.

If the performance of an operation is described herein as being “based on” one or more factors, it is to be understood that the performance of the operation can be based solely on such factor(s) or can be based on such factor(s) along with one or more additional factors. Thus, as used herein, the term “based on” should be understood to be equivalent to the term “based at least on.”

Still further, it should be noted that the drawings/figures are not drawn to scale unless otherwise noted herein.

Numerous exemplary embodiments are now described. Any section/subsection headings provided herein are not intended to be limiting. Embodiments are described throughout this document, and any type of embodiment may be included under any section/subsection. Furthermore, it is contemplated that the disclosed embodiments may be combined with each other in any manner. That is, the embodiments described herein are not mutually exclusive of each other and may be practiced and/or implemented alone, or in any combination.

### II. Example Embodiments

Electronic prior authorizations are authorization requests (e.g., for a medication) that are sent electronically over a network from one entity to another. In conventional systems, an electronic prior authorization requires answering a set of questions sent electronically (e.g., from a PBM/payer), which are filled in manually (e.g., by a provider). The process typically requires an entity (e.g., a provider) to access an electronic health record and/or other sources of clinical information over a network to fill out answers to the questions, from which a determination can be made (e.g., by the PBM/payer) whether the authorization should be approved. Where billions of prescriptions need to be evaluated every year for a potential prior authorization, inefficiencies can result based on the systems and/or networks used for completing, reviewing, and/or approving the authorizations.

Accordingly, the pharmacy benefits electronic prior authorization process remains highly manual. While the National Council for Prescription Drug Programs (NCPDP) standard exists for transmitting the question sets for an Electronic Prior Authorization, the standard is inadequate with respect to codification of questions and answers such that questions are automatically completed based on electronic health information. In addition, the NCPDP standard does not provide support for a suitable model where a prior authorization (PA) could be automatically approved based on clinical data pulled from an electronic health record (EHR), nor does it provide support for a partially suitable model where a PA could be sometimes approved based on clinical data pulled from the EHR. In addition, EHRs and pharmacy benefits manager (PBM) PA systems are typically not designed to partially or fully automate the PA process. To do this at scale within existing systems and standard, an intelligent transaction filtering model is disclosed to manage the potential volume of prescriptions that may require a PA (e.g., several billion or more per year).

Methods, systems, and apparatuses are described for filtering a transaction over a network, according to embodiments. In an example system, a transaction request is retrieved over a network. A model that is configured to predict a likelihood associated with automatic adjudication of transactions is accessed. Based at least on the transaction request and the model, a likelihood that the transaction request can be adjudicated automatically is determined. If the likelihood that the transaction request can be adjudicated automatically is above a threshold, one or more actions are performed. In one example, an indication that at least a portion of the transaction request can be adjudicated automatically is generated. In another example, an automatic adjudication is generated indicating whether the transaction request should be approved. In response to generating the indication or automatic adjudication, the transaction request and the indication or the automatic adjudication are forwarded over the network to an endpoint associated with the transaction request.

In this manner, techniques allow for filtering transaction requests such that those requests that are suitable for automatic approval can be readily identified. For instance, when the likelihood of automatic adjudication is above a threshold, data (e.g., clinical data) associated with a transaction request can be retrieved automatically to identify the appropriate information (e.g., answers to questions) associated with the request to provide to another entity (e.g., a PBM) where an approval or denial decision may occur. In another example, when the likelihood is above the threshold, the appropriate clinical data be retrieved to automatically adjudicate the transaction request (e.g., by approving or denying the request, or recommending an approval or denial). Where the likelihood is not above the threshold, the transaction request is deemed not suitable for an automatic adjudication, and such data therefore need not be retrieved.

The example techniques and embodiments described herein may be adapted to various types of systems and devices, for example but without limitation, computing systems (e.g., computers/computing devices such as desktops, laptops, etc., and servers, enterprise computing systems, etc.), communication devices (e.g., cellular and smart phones, tablets, smart watches, etc.), and/or the like, that communicate information and data that relates to, without limitation, electronic prior authorizations, in different ways, in accordance with embodiments. For instance, computing systems that communicate over a network approval of an electronic prior authorization, may be configured according to the described embodiments and techniques.

Accordingly, example embodiments relate to systems and methods in which an intelligent determination is made whether a system (e.g., a transaction approval system) is likely to automatically approve a PA in certain instances, such as where sufficient clinical data is available for the approval. In other instances, such as where sufficient clinical data is not available or the transaction cannot otherwise be automatically approved, the transaction (i.e., the electronic prior authorization) may continue by forwarding the request unaltered (e.g., to a computing device of a user responsible for reviewing PAs). In some implementations, data from a prescription (e.g., a new prescription transaction), real-time prescription benefits transactions, prior authorization transactions, formulary data, and prescription change transactions that indicate that a prior authorization is required may be used to train a machine-learning model in accordance with various implementations. Illustrative techniques for training the machine learning model will be described in greater detail below.

While the embodiments herein may be described with respect to various computing systems and implementations as conceptual and/or illustrative examples for descriptive consistency, other types of electronic and communication devices and implementations are also contemplated for implementing the disclosed techniques. It is contemplated herein that in various embodiments and with respect to the illustrated figures of this disclosure, one or more components described and/or shown may not be included and that additional components may be included.

Additionally, while the embodiments herein may be described with respect to electronic prior authorizations, they are not so limited, as noted above. However, embodiments may be exemplarily described in various contexts where requests are transmitted over a network for approval, as illustrative of the more general techniques that encompass the embodiments herein. Other embodiments contemplated herein may be realized, based on the illustrations and examples provided herein.

The techniques described herein have numerous advantages, including but not limited to reducing the amount of data transmitted over a network to one or more computing devices. For instance, by filtering transactions (e.g., data packets) that are likely to be approved automatically, those transactions need not be transmitted over a network to a computing device where manual review typically takes place. Rather, such transactions may be intercepted at a server or other computing device, where the transaction may be automatically approved (including recommending for approval) without transmission thereof to other devices. Furthermore, given that the manual review process typically requires accessing other information over a network (e.g., a patient health record or other information), such additional data need not be transmitted to the computing device where manual reviews take place for transactions that are approved automatically. As a result, networking resources may be preserved through implementation of the disclosed techniques. For instance, processing each new prescription and/or prior authorization request for automatic adjudication may potentially utilize substantial compute power. In accordance with disclosed techniques, such requests may be intelligently filtered to reduce network traffic because requests that do not have a minimum probability of success are not processed in various implementations.

In addition, processing resources (e.g., resulting from accessing a received transaction and accessing other related information during the approval process) and/or storage resources (e.g., due to storage of received information, such as the transaction and/or health records) may also be conserved, thereby resulting in an overall performance improvement in such a computing device. Still further, disclosed techniques may also implement models using artificial intelligence (AI) or machine learning techniques to determine whether a transaction can be automatically approved, or is likely to be automatically approved, and therefore should be intercepted. Such models may be specifically trained as described herein to make such a determination in an efficient manner, which can further improve the performance of the system in which the models are utilized. Thus, in addition to the reducing the computing resources as described above, techniques implementing trained models as described herein further improve the utilization of processing resources. Other advantages and benefits will be described in greater detail below.

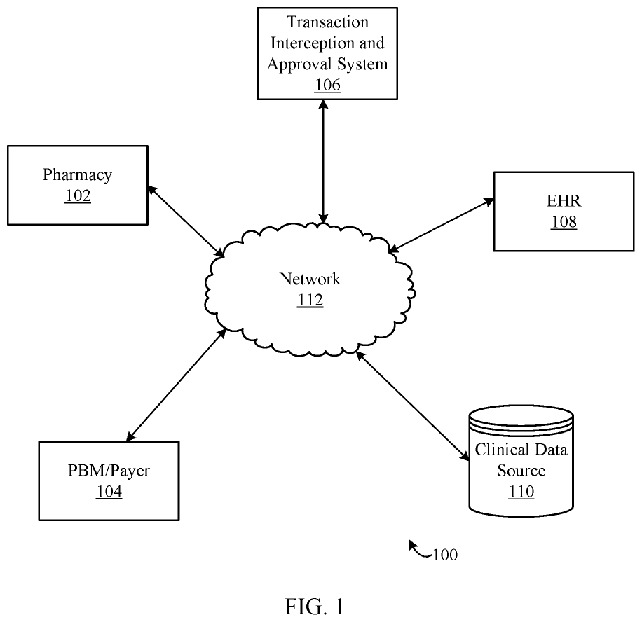

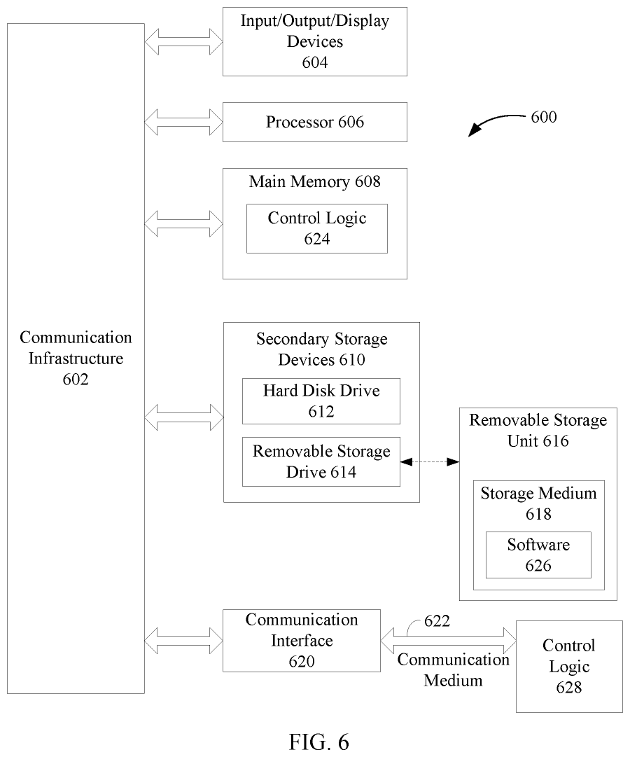

Systems and devices may be configured in various ways for intelligent filtering of a transaction over a network, according to embodiments. For instance, in FIG. 1, a block diagram of a system 100 is shown for intelligent filtering of a transaction over a network, according to an example embodiment. System 100 includes a pharmacy 102, a pharmacy benefits manager (PBM)/payer 104, a transaction interception and approval system 106, an electronic health record (EHR) 108, and a clinical data source 110. Each of pharmacy 102, PBM/payer 104, transaction interception and approval system 106, EHR 108, and/or clinical data source 110 may be implemented on and/or comprise one or more processing devices, such as a computing device and/or a server. An example of such devices is described below with respect to FIG. 6. In implementations, each of pharmacy 102, PBM/payer 104, transaction interception and approval system 106, EHR 108, and/or clinical data source 110 are coupled via a network 112. In embodiments, one or more of any component shown in system 100 of FIG. 1 may be present in various implementations.

Pharmacy 102 comprises one or more computing devices of an entity which receives a prescription for fulfillment. In some examples, pharmacy 102 may receive an approval of a transaction (e.g., an approval of an electronic prior authorization from a PBM) as described herein and dispense a medication according to the transaction.

PBM/payer 104 comprises one or more computing devices of an entity that may process prior authorization transactions in some implementations. In some examples, PBM/payer 104 may cause a transaction to be approved, denied, or have some other status, and cause a health record to be updated. After approving an authorization, an indication of the approval may be transmitted to a pharmacy for fulfillment and/or a prescriber.

Transaction interception and approval system 106 comprises one or more computing devices of an entity that intercepts transaction requests (such as electronic prior authorizations) over a network to determine whether the transaction request may be suitable for automatic adjudication and/or whether the request can be automatically approved (or recommended for approval). For instance, transaction interception and approval system 106 may determine a likelihood that the sufficient information (e.g., clinical data) is available to automatically adjudicate the transaction request to determine whether it should be approved or denied. In various examples, an automatic adjudication of a transaction request indicates that a requisite set of evidence (e.g., data retrieved from a clinical data source or other source) was retrieved such that a transaction request is able to be approved without human intervention (or with a reduction in human intervention).

In one example, the likelihood represents a likelihood that the transaction request would be approved automatically. If the likelihood is above a threshold (e.g., indicating that the sufficient information exists or that the transaction is likely to be approved), transaction interception and approval system 106 may apply one or more rules (e.g., machine learning-based rules) and/or access an EHR of an associated patient to determine whether the transaction should be automatically approved or is suitable for an automatic adjudication. For instance, transaction interception and approval system 106 may automatically retrieve a set of data (e.g., clinical information) associated with the transaction request and associate such data with questions corresponding to the request, and send this information to a separate entity (e.g., the PBM) for adjudicating the request. In another example, transaction interception and approval system 106 may generate an automatic adjudication (which includes a recommended adjudication) of the request based on the set of data. If the likelihood is below a threshold (indicating that the transaction request is not suitable for an automatic adjudication), transaction interception and approval system 106 may allow the transaction to proceed in a normal (e.g., unaltered) fashion, by allowing the transaction to be transmitted to an endpoint associated with the transaction request, such as by transmitting the unaltered request to PBM/payer 104 or pharmacy 102. Additional details regarding the operation and functionality of transaction interception and approval system 106 will be described in greater detail below.

EHR 108 comprises an electronic health record of a patient associated with the transaction. EHR 108 may be stored in any suitable fashion (e.g., as a file or collection of files), and may contain any medical information, such prior prescription history, health records, etc. of an individual.

Clinical data source 110 comprises one or more storage devices for storing medical information for one or more individuals. In some implementations, clinical data source 110 comprises one or more databases or other data sources that store patient information, such as EHRs of patients. Any number of clinical data sources may be present in implementations.

Network 112 may comprise at least one network and/or direct connection (i.e., a communication link), or any combination thereof. That is, network 112 may be any combination of the Internet, the “cloud”, direct communication links, business and/or enterprise networks, and/or the like. In examples, network 112 is configured to communicatively couple pharmacy 102, PBM/payer 104, transaction interception and approval system 106, EHR 108, and/or clinical data source 110 to each other. In some embodiments, such as cloud-based implementations, one or more of the foregoing entities may be implemented as a service or application via network 112.

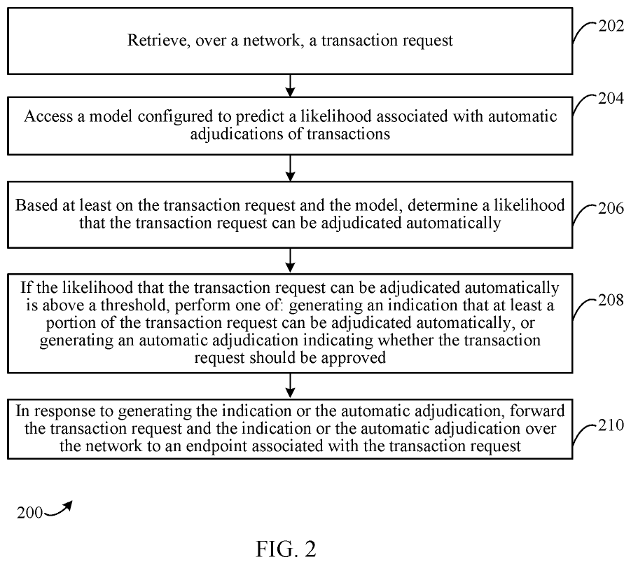

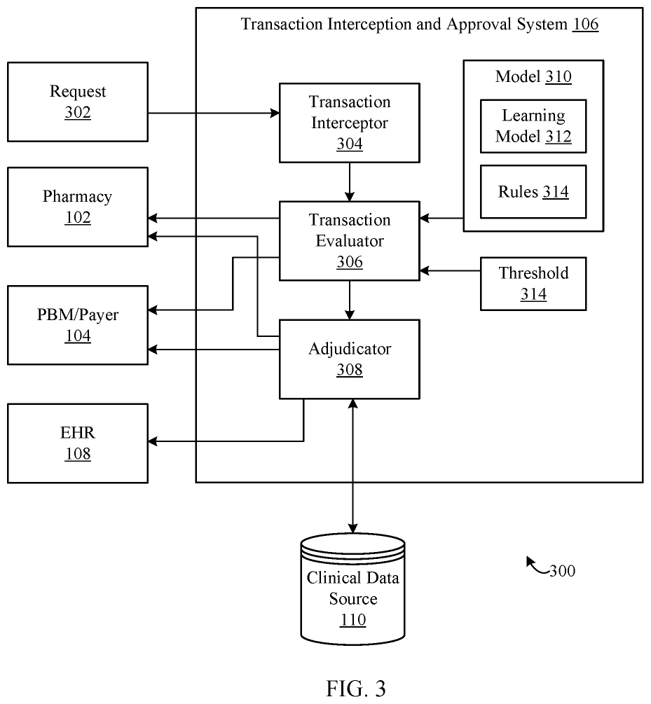

Transaction interception and approval system 106 may operate in various ways in examples. For instance, transaction interception and approval system 106 may operate according to FIG. 2. FIG. 2 shows a flowchart 200 of a method for intelligent filtering of a transaction over a network, in accordance with an example embodiment. Further structural and operational examples will be apparent to persons skilled in the relevant art(s) based on the following descriptions. For illustrative purposes, flowchart 200 and transaction interception and approval system 106 are described as follows with respect to FIG. 3.

FIG. 3 shows a block diagram of a transaction interception system 300, according to an example embodiment. As shown in FIG. 3, system 300 includes an example implementation of transaction interception and approval system 106, pharmacy 102, PBM/payer 104, EHR 108, and clinical data source 110. System 300 also includes a request 302 retrieved by transaction interception and approval system 106. As shown in FIG. 3, transaction interception and approval system 106 comprises a transaction interceptor 304, a transaction evaluator 306, an adjudicator 308, a model 310, and a threshold 314. Model 310 includes a learning model 312 and a set of rules 314. Additional details regarding the operation of system 300 are provided below.

Flowchart 200 begins with step 202. In step 202, a transaction request is retrieved over a network. For instance, with reference to FIG. 3, transaction interceptor 304 is configured to retrieve request 302 over a network 112. In various embodiments, request 302 comprises a request for authorization of a transaction. For instance, the request may comprise a request for prior authorization of a medical good or service. As used herein, a medical good comprises a product or other tangible item used for a medical purpose. A medical service comprises an activity or benefit provided by one entity to another for a medical purpose. Examples of medical goods or services include, but are not limited to, a medication for a patient (e.g., based on the patient's insurance), a prior authorization of medical services or treatments, a prior authorization for medical equipment, or other types of prior authorizations for a patient for receiving medical care. While example embodiments are disclosed with respect to such prior authorizations, it should be understood that the disclosed techniques may be implemented in other environments where authorizations may be needed prior to providing services or products.

In example embodiments, transaction interceptor 304 retrieves request 302 by intercepting the request from a transmitting source (e.g., an EHR, a provider, etc.). For instance, transaction interceptor 304 may intercept the transaction request over the network before an endpoint associated with the transaction request (e.g., a PBM/payer, or pharmacy) receives the request. In another example, transaction interceptor 304 retrieves request 302 from an entity different than the transmitting source, such as from a PBM/payer, a pharmacy, or other entity (e.g., an endpoint associated with the request). As described in greater detail below, retrieval of the transaction request may be performed in various ways, such as by intercepting an initiation of a prior authorization or intercepting a new order for a medical good or service (such as a prescription). These examples are only illustrative, and other types of transaction questions are also contemplated herein.

In step 204, a model configured to predict a likelihood associated with automatic adjudications of transactions is accessed. For instance, with reference to FIG. 3, transaction evaluator 306 accesses model 310 to predict a likelihood that request 302 can be adjudicated automatically. In examples, model 310 comprises any one or more of machine learning model 312 or a set of rules 312 (e.g., predefined rules) associated with automatic adjudications of transactions (e.g., rules that define how transaction authorizations are automatically processed). For examples, model 310 may define the types of requests that can be adjudicated automatically, what types of clinical data would be obtained, whether such clinical data is likely to be present in clinical data source 110, or various other parameters as described in greater detail below. Rules 312 include any set of requirements that may be predefined that dictate whether and how a prior authorization should be approved, such as questions that should be answered, clinical data (e.g., diagnoses, treatments, symptoms, conditions, etc.) that support approval of a prior authorization, or other requirements for approval.

In step 206, based at least on the transaction request and the model, a likelihood that the transaction request can be adjudicated automatically is determined. For instance, with reference to FIG. 3, based at least on application of model 310 to request 302, transaction evaluator 306 may determine a likelihood that request 302 can be automatically adjudicated. The automatic adjudication may comprise, for instance, an approval or denial of request 302 or a recommendation for an approval or denial of request 302. For instance, in some examples, the transaction request itself may not be fully approved or denied by transaction interception and approval system 106, but may comprise a recommendation for approval or denial (e.g., based on additional data, rules, and/or requirements that may not be provided to transaction interception and approval system 106). In such an example, the recommendation may be provided to another entity (e.g., PBM/payer 104) where an appropriate decision may be made (such as approval or denial of a request based at least on the generated recommendation). In other examples, the automatic adjudication may comprise one or more tasks associated with evaluating request 302, such as populating a question set associated with request 302, identifying additional questions, and/or partially approving or denying (or recommending a partial approval or denial) the request.

In some implementations, the automatic adjudication comprises answering a portion (e.g., a subset or all) of the questions from an entity (e.g., the PBM/payer) relating to the transaction request (and optionally including or referencing evidence for each answer from a clinical data source), even if a transaction request is not approved or denied (or recommended for approval or denial). In such an example, the remaining questions may be transmitted to a provider for answering the one or more questions that were unanswered by transaction interception and approval system 106. In this manner, even if a transaction request is not approved or denied (or recommended for approval or denial), partial resolution of a transaction request may nevertheless reduce the amount of back and forth between various entities (e.g., a provider and a PBM, or a provider and a clinical data source), thereby reducing computing resources (e.g., network resources).

As an illustration, if an entity (e.g., PBM/payer 104) defines the type of clinical data (e.g., medical conditions, symptoms, diagnoses, etc.) that is needed for approval of a prior authorization, transaction interception and approval system 106 may return answers to any of the questions in an associated question set (e.g., by returning a “yes,” “no,” or other appropriate answer), and/or returning the clinical data evidence that is needed to adjudicate the prior authorization. In this case, the PBM could use the clinical data evidence that is identified by transaction interception and approval system 106 to eliminate questions that would otherwise be provided to the provider (given that the PBM now possesses the needed information), and/or the PBM could have enough information to approve the PA without providing additional questions to the provider. As noted above, such techniques allow for reduction in the back-and-forth between various entities involved in the approval process, which can conserve computing resources.

In another implementation, the request may be initiated upon a pharmacy receiving a prescription. Once the pharmacy receives a prescription, the pharmacy may send a PA initiation request to PBM/payer 104. In such a scenario, the PA could be requested via various types of systems that are part of different networks and/or systems (e.g., systems maintained and/or operated by entities different from the operator of transaction interception and approval system 106). Once the PBM receives the request, the PBM may send a signal (e.g., a request) to transaction interception and approval system 106 to obtain a set of clinical data corresponding to the PA initiation request (e.g., evidence relating to the requested prescription). In response, transaction interception and approval system 106 may generate an indication that the PA initiation request is suitable for automatic adjudication. Transaction interception and approval system 106 return the indication, which may include the clinical data and/or an appropriate set of answers associated with the PA initiation request, to the PBM. In this illustrative scenario, the PBM may then use the received information to return a truncated question set or automatically approve the PA with the originating system (which may be operated by a different entity). Thus, while some example embodiments are disclosed herein in which transaction interception and approval system 106 may automatically adjudicate a transaction (or recommend an adjudication), examples are not so limited. Rather, some implementations allow for transaction interception and approval system 106 to automatically identify, retrieve, and return an appropriate set of data (e.g., clinical data) to the PBM where an adjudication for the request can be made.

In various embodiments, transaction evaluator 306 does not access clinical data source 110 to determine the likelihood that request 302 can be automatically adjudicated. Rather, transaction evaluator 302 may determine the likelihood based on the content of request 302 and evaluating the request based on learned behaviors of prior adjudications to determine whether the request should be further processed by transaction interception and approval system 106. In this manner, transaction evaluator 306 may perform a preliminary evaluation of request 302 that utilizes fewer computing resources (such as reduced network resources by avoiding the access of clinical data source 110 and processing resources associated therewith) before performing a more detailed evaluation of the request.

In another example, transaction evaluator 306 may determine the likelihood based on an analysis of the capabilities of a source technology platform, and/or an interoperability characteristic of a source technology platform. The capabilities of the source technology platform can include capabilities of a data source providing clinical data (e.g., clinical data source 110 and/or EHR 108), which may include whether the data source can provide data, the quality of the data, a level of comprehensiveness of the data, the speed at which data is obtained, and/or the types of data that are available and/or retrievable to support a transaction request. Interoperability characteristics of the source technology platform can include information associated with an interoperability of the clinical data source and the system retrieving data from the clinical data source (e.g., whether and how communications can be carried out between the systems, which may be operated by different entities, whether the payer has data rights to access data from a different platform based on an interoperability characteristic, etc.).

In step 208, if the likelihood that the transaction request will be adjudicated automatically is above a threshold, one or more actions are performed, which include generating an indication that at least a portion of the transaction request can be adjudicated automatically or generating an automatic adjudication indicating whether the transaction request should be approved. For instance, with reference to FIG. 3, transaction evaluator 306 may determine whether the likelihood that request 302 can be adjudicated automatically is above threshold 314. Threshold 314 may indicate, for instance, a minimum likelihood that should be present for transaction interception and approval system 106 to further evaluate request 302.

If the likelihood is above threshold 314, transaction evaluator 306 may generate an indication that at least a portion of the transaction request can be approved automatically or adjudicator 308 may generate an automatic adjudication indicating whether request 302 should be approved. In various examples, the indication may indicate that any part of the transaction request can be processed automatically. For instance, in one implementation, transaction evaluator 306 may identify and retrieve data required to process a part of, or all of the transaction request (e.g., the prior authorization). In one example, transaction evaluator 306 is configured to retrieve evidence associated with the transaction request (e.g., answers to one or more questions of a question set, where such answers are based on clinical or other data retrieved from a data source). In such an example, transaction evaluator 306 may generate an indication that includes such evidence associated with the transaction request, such that the evidence may be provided to a different entity (e.g., where a different entity is responsible for the adjudication), such as PBM/payer 104.

In another example, adjudicator 308 may generate an automatic adjudication of the transaction request, which can include an automatic approval or denial of request 302, or a recommendation for an approval or denial of request 302 (which may then be provided to another entity, as described below). In examples, adjudicator 308 may access other data sources, such as clinical data source 110 (which may contain records associated with a patient that is the subject of request 302), to generate the automatic adjudication for request 302.

Transaction evaluator 306 and/or adjudicator 308 may generate the indication and/or the automatic adjudication in various ways. In one implementation, transaction evaluator 306 and/or adjudicator 308 may generate the indication and/or the automatic adjudication based on an analysis of clinical data obtained from clinical data source 110 (e.g., whether evidence in the data source supports approval of the PA), demographic data, or payer data associated with a patient. Demographic data can include demographic information for a given patient, such as age, gender, location, etc. Payer data can include payment, benefits, and/or claims history for a patient, such as a history of past claims, past PA approvals or denials, insurance coverage information, etc.

If the likelihood of automatic adjudication of request 302 is not above threshold 314 (indicating that the transaction request is not suitable for an automatic adjudication), the request may be forwarded (e.g., unaltered) to another destination, such as an endpoint associated with the transaction request that may include pharmacy 102, PBM/payer 104, or another entity where the request may be processed by other means (e.g., by a manual review or other techniques).

In step 210, in response to generating the indication or the automatic adjudication, the transaction request and the indication or the automatic adjudication are forwarded over the network to an endpoint associated with the transaction request. For instance, with reference to FIG. 3, in response to generating the indication, transaction evaluator 306 may forward the indication (e.g., answers to questions) and/or the transaction request to an endpoint over the network to an endpoint, such as a PBM/payer 104 where a determination is to be made on approval or denial of the transaction request. In some implementations, PBM/payer 104 may implement one or more techniques as described herein to automatically adjudicate the transaction request based on the evidence provided by transaction evaluator 306.

In another example, in response to generating the automatic adjudication, adjudicator 308 may forward the automatic adjudication and/or request 302 to an endpoint associated with request 302. The endpoint may comprise a destination as specified in the original request 302, or may comprise a different destination. In illustrative examples, the endpoint may comprise pharmacy 102, PBM/payer 104, and/or EHR 108 (or another entity not expressly shown).

Various additional embodiments for intelligent filtering of a transaction request over a network in the context of the system and flowchart described above are provided below.

In embodiments, a machine learning model (e.g., learning model 312) may be used to compute a probability (e.g., a likelihood) that a transaction can be automatically adjudicated (e.g., approved or denied). At a design-time of the machine learning model (e.g., prior to deployment), the model may be configured to analyze questions from question sets (e.g., questions that may be included within a given electronic prior authorization). In some implementations, during design time, semantic meanings of each question are determined and/or a determination is made with respect to which questions from the sets are computable. In some other embodiments, a determination made be made with respect to how questions may be answered (e.g., whether an answer may be obtained from an EHR, a clinical data source, or some other source), and whether such data sources are likely to contain sufficient information to determine answers to those questions. The model may also be configured to cluster related questions that can be answered using rules applied to structured clinical data (e.g., from clinical data source 110). In some embodiments, the model may determine prior authorizations for insurance and/or medication that can be automatically adjudicated using rules and/or structured clinical data. In some further embodiments, the model may also determine a likelihood that data sources are available that contain adequate clinical information to answer questions from questions sets.

Training data for training learning model 312 may include various types of information, including but not limited to information associated with patient insurance plans, medications, providers, prior authorization transactions, real-time prescription benefits, prescription change (RxChange) messages, and/or formulary data. In embodiments, patient insurance plan information may include a National Council for Prescription Drug Programs (NCPDP) Program Identification Number (BIN) and/or Processor Control Number (PCN). Medication information may include NDC and/or other codes (e.g., RXNorm) codes, quantity of medication, a length of supply (e.g., days supply) of medication. Provider information may include a National Provider Identifier (NPI), or other provider identifier, practice location of a provider, clinical data source associated with a provider, and/or an EHR used by a provider. Prior authorization transaction (e.g., electronic prior authorizations) information may include question sets, answers, and/or outcomes. Real-time prescription benefit information may include NDC information, pharmacy information, quantity, length of supply, payer information, an indication of whether a prior authorization is needed, quantity limits, and/or step therapy information. Prescription change messages may include prior authorization required status and/or NDC and prescription information. Formulary data may include NDCs with prior authorization flags. In another example, training data may include learning (e.g., as a duration) how often a PA is needed to be reauthorized. These examples are only illustrative, and other types and/or categories of information may be used for training a model as described herein.

In embodiments, the model may compute the probability of successful automatic adjudication of the prior authorization at runtime to enable intelligent transaction filtering. In some implementations, the probability may be computed for each prescription (e.g., new prescription) sent electronically between the EHR and pharmacy and/or from the EHR to the PBM.

In implementations, the probability may be computed based on a combination of different probabilities. For instance, the model may compute a probability that a prior authorization is required. In another implementation, the model may compute a probability that if a prior authorization is required, it may be denied based on other conditions, such as quality limits, days supply limits, formulary status, and/or other benefit design information. In another implementation, the model may compute a probability of required clinical data being available to satisfy the computable rules. In other words, transaction interception and approval system 106 may be configured to constantly monitor transactions that are being processed by the system and determine which transaction requests are able to be processed successfully and/or unsuccessfully, and retrain the model based on ongoing learning.

In some embodiments, transaction interception and approval system 106 may periodically process transactions with a relatively low probability of satisfying any of the aforementioned scenarios to determine if the source conditions have changed and to update the model. For instance, if an EHR that previously did not provide sufficient information (or was insufficient in other regards, such as due to compatibility issues) is later updated such that it provides improved and/or more comprehensive information, the model may be updated so that transaction requests that may rely on this EHR may have a higher chance of being processed by transaction interception and approval system 106.

Accordingly, examples described herein may be implemented for generating a machine-learning model to identify one or more prior authorization requests that meet a threshold for further processing (e.g., in a transaction approval system, such as transaction interception and approval system 106). In an example system, transaction interception and approval system 106 comprises various components for generating such a model. In one example, transaction interception and approval system 106 comprises at least a vector generator, a vector combiner, and/or a model generator. The vector generator may retrieve prior authorization training data corresponding to tracked prior authorizations (e.g., historical prior authorizations). The vectors may be generated in various ways. In examples, the vector generator may generate a plurality of vectors based at least on one or more of the prior authorization training data, a prior authorization history, a prescription history, a data source providing clinical data, or any other suitable set of information associated with prior authorization approvals. The vector combiner may be configured to generate combined prior authorization vectors based at least on the plurality of vectors. The model generator may be configured to generate a recommendation model using a machine-learning algorithm (e.g., a supervised algorithm) based at least on the combined prior authorization vectors. In examples, the recommendation model is configured to identify a likelihood that a retrieval of clinical data will satisfy a set of rules configured for the least one of a payer or medication identified in a transaction request (e.g., request 302).

The vector generator may comprise any number of components for generating different types of vectors. In examples, the vector generator comprises one or more of a result vector generator, a provider vector generator, a data source vector generator, a medical goods or services vector generator, a payer vector generator, a questionset vector generator, a diagnosis code vector generator, or a clinical data vector generator. The result vector generator may be configured to generate prior authorization result vectors corresponding to outcomes based on the prior authorization history. The provider vector generator may be configured to generate provider vectors corresponding to a requesting provider based on the prior authorization history. The data source vector generator may be configured to generate data source vectors corresponding to the data source providing clinical data. For instance, the data source vectors may comprise information associated with technology capabilities of a data source providing clinical data (e.g., clinical data source 110), which may include whether the data source can provide data, the quality of the data, a level of comprehensiveness of the data, the speed at which data is obtained, the types of data that are retrievable, and/or an interoperability characteristic of the clinical data source and the system retrieving data from the clinical data source (e.g., whether and how communications can be carried out between the systems). In some examples, the data source vectors also include data relating to a data source providing administrative data, such as past claims history, past prior authorization approvals or denials for a patient, and/or other similar types of data. The medical goods or services vector generator may be configured to generate medical services vectors corresponding to a medical goods or service based on a a prior authorization history of the same or similar medical good(s) or service(s) (e.g., a medication requested based on the prior authorization history of the same or similar medications). The payer vector generator may be configured to generate payer vectors corresponding to a payer based on the prior authorization history. The questionset vector generator may be configured to generate questionset vectors corresponding to a questionset based on the prior authorization history. The diagnosis code vector generator may be configured to generate diagnosis code vectors corresponding to a diagnosis code set based on the prior authorization history and a medical goods or services history (such as a prescription history). The clinical data vector generator may be configured to generate clinical data vectors that relate to labs, diagnostic tests, medical evaluations, or other clinical data that may be relevant to predicting whether any part of a prior authorization can be adjudicated or approved automatically. These examples are only illustrative, and it should be understood that other vectors may be generated in accordance with the disclosed techniques, where such vectors relate to information that can be used to predict whether a prior authorization can be automatically approved.

The vector combiner may be configured to generate prior authorization vectors based at least on the plurality of vectors that were generated. For instance, the vector combiner may generate prior authorization vectors based on any combination of the result, provider, data source, medical goods or services, payer, questionset, diagnosis code vectors, or clinical data vectors (or any other vectors not expressly described herein). In examples, the model generator may be configured to retrieve prior authorization training data corresponding to tracked prior authorizations. The model generator may also generate a recommendation model using a supervised machine-learning algorithm that receives the prior authorization training data and the prior authorization vectors as inputs, and identify (e.g., as an output) a likelihood that a retrieval of clinical data will satisfy a set of rules configured for the least one of the payer or medication requested. In this manner, the recommendation model may generate a prediction indicative of whether a prior authorization is likely to be automatically adjudicated (e.g., approved).

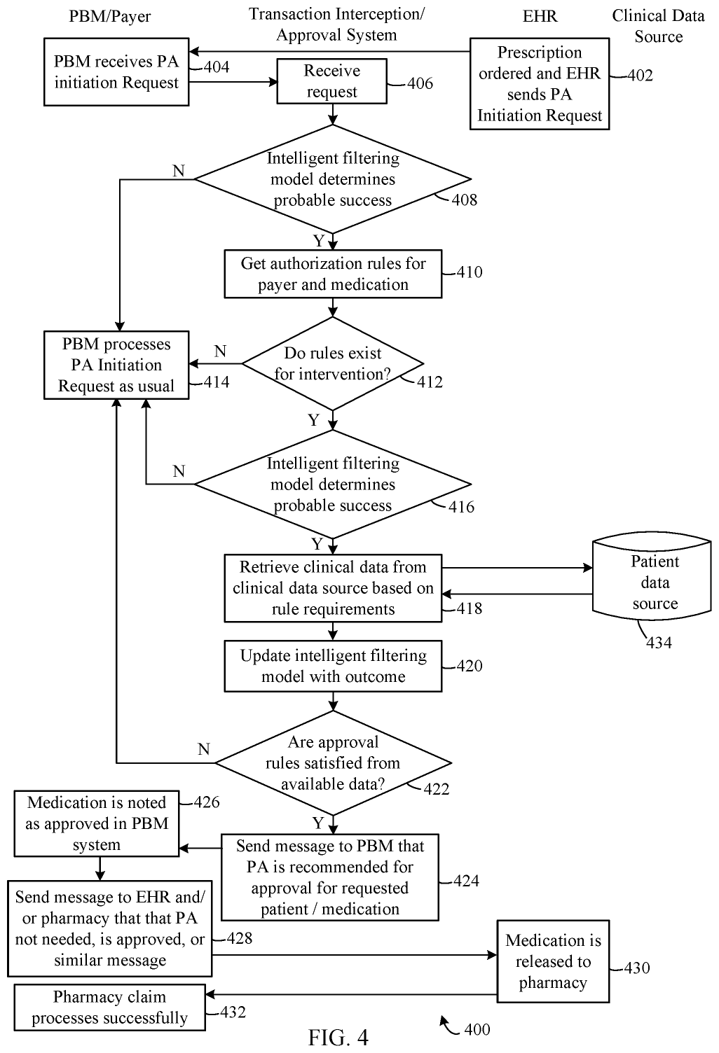

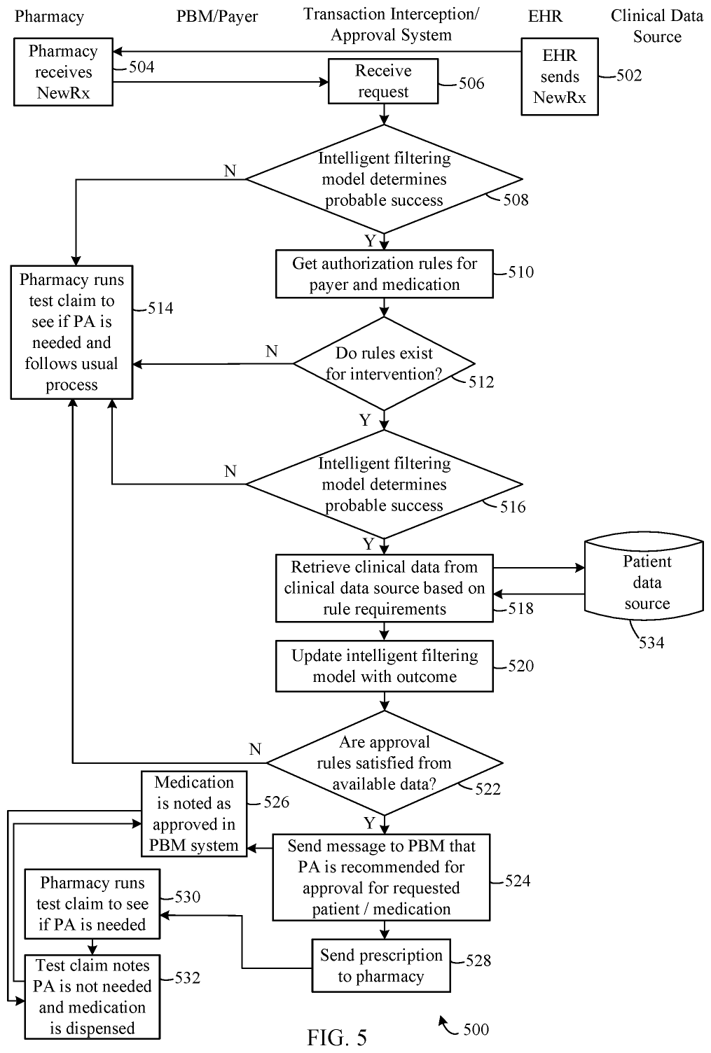

In embodiments, transaction interception and approval system 106 may automatically determine whether to approve a transaction. In an implementation, transaction interception and approval system 106 may configure rules such that a prior authorization can be adjudicated by applying clinical data from patient record to the rules. In examples, the rules may be specified by the PBM or learned using the model trained by reviewing existing transactions/question sets and outcomes. Once rules are configured, transaction interception and approval system 106 may automate the prior authorization process in various ways. Non-limiting examples are provided below with respect to FIG. 4 and FIG. 5.

FIG. 4 shows a flowchart 400 for a first illustrative implementation of a transaction interception, in accordance with an example embodiment. Further structural and operational examples will be apparent to persons skilled in the relevant art(s) based on the following descriptions. Flowchart 400 is described as follows with respect to system 100 of FIG. 1 and/or system 300 of FIG. 3. One or more of the steps of flowchart 400 may be performed by PBM/payer 104, transaction interception and approval system 106, EHR 108, and/or clinical data source 110.

Flowchart 400 begins at step 402. In step 402, a prescription is ordered and EHR 108 sends a PA initiation request to PBM/payer 104. In some examples, the PA is initiated at the EHR. In other examples, the PA may be initiated at another entity not expressly shown.

In step 404, PBM/payer 104 receives the PA initiation request. In step 406, transaction interception and approval system 106 receives the PA initiation request. In some implementations, transaction interception and approval system 106 receives the request from PBM/payer 104. In other examples, transaction interception and approval system 106 may intercept the PA after initiation (e.g., by intercepting a communication between EHR 108 and PBM/payer 104). In some examples, the PA initiation request received by transaction interception and approval system 106 may comprise a set of questions associated with the requested prior authorization. In other examples, transaction interception and approval system 106 may access the set of questions locally (e.g., local to transaction interception and approval system 106) and/or remotely upon receiving the PA initiation request.

In step 408, an intelligent filtering model determines a probability of success of adjudicating the PA initiation request. For instance, transaction interception and approval system 106 may apply intelligent transaction filtering as described herein (e.g., by applying model 310) to determine if the request should be processed by transaction interception and approval system 106 (e.g., to determine whether the transaction is suitable for automatic adjudication of the PA). If the probability is above a threshold, the flow proceeds to step 410. If the probability is not above the threshold, the flow proceeds to step 414. For instance, by utilizing an intelligent filtering model to filter out transactions that are not suitable for automatic adjudication and processing only those transactions that are suitable, computing resources can be preserved. For example, the retrieval (over a network) and processing (using a CPU) of clinical data can be limited to only a subset of transactions that are deemed suitable for automatic adjudication.

In various examples, step 408 is performed using a first set of filters of the intelligent filtering model. For example, in step 408, any one or more of the techniques described herein with respect to application of model 310 can be applied to determine whether further processing of the transaction request should be performed (e.g., whether the transaction request is suitable for an automatic adjudication, whether data associated with the transaction request should be retrieved from a clinical data source, etc.), or whether the request should be processed in other ways (e.g., as described with respect to step 414). For instance, if application of the intelligent filtering model indicates a likelihood automatic adjudication above a threshold, additional steps can be performed to process the transaction (e.g., ask the payer for the question set, perform additional transaction filtering, retrieve clinical data, etc.).

In step 410, authorization rules for a payer and/or medication associated with the request are obtained. For example, transaction interception and approval system 106 may access a set of rules for determining whether the request should be processed by transaction interception and approval system 106. For instance, the rules can include a combination of medication (e.g., NDC or equivalent code) and plan information to determine if there is a potential for automatically approving the PA and/or computable, codified rules that evaluate clinical data against the PA requirements. In step 412, a determination is made whether rules exist for intervention (e.g., rules for the NDC/plan combination). If rules exist for intervention, the flow proceeds to step 416. If rules do not exist for intervention, the flow proceeds to step 414. At step 414, the PBM processes the PA initiation request as usual (e.g., by a manual review or by other means in the ordinary course). In some examples, an outcome determined as a result of processing the PA initiation request as usual may be provided to transaction interception and approval system 106 in order to train model 310 to improve its accuracy.

At step 416, transaction interception and approval system applies an intelligent filtering model (e.g., model 310) an additional time (e.g., by analyzing more information associated with the request) to determine a probability of success of automatic adjudication. If the probability is above a threshold the flow proceeds to step 418. If the probability is not above the threshold, the flow proceeds to step 414.

In various examples, step 416 may perform an additional filtering of transactions by applying a second set of filters of the intelligent filtering model that differs from the first set of filters described above. For example, the second set of filters may include one or more of filters from the first set, as well as one or more additional filters (e.g., filters based on payer rules, data requests, or other information retrieved from a question set or from some other source that indicates the type of information or evidence needed to analyze the PA initiation request).

In step 418, clinical data is retrieved from a clinical data source (e.g., patient data source 434, which is an example implementation of clinical data source 110) based on rule requirements. For instance, if a rule exists for an NDC/plan combination and/or the probability of adjudication is above a threshold, clinical data may be obtained from EHR 108 and/or patient data source 434 (or any other source which contains patient medical information and/or history, accessed via any suitable means or system).

In step 420, an outcome is determined (e.g., an adjudication of the request, which may comprise a recommended adjudication), and the intelligent filtering model is updated with the outcome. For instance, model 310 may be updated with the outcome to improve its accuracy. As an example, model 310 may be retrained and/or updated in a manner such that it may determine (e.g., for a particular provider or clinical data source) that quality data can (or cannot) be retrieved for evaluation of a request.

In step 422, a determination is made whether approval rules are satisfied from available data. For instance, transaction interception and approval system 106 may evaluate the clinical data against computable, codified rules to determine if relevant rules are satisfied to approve medication (e.g., approve the PA). If relevant rules are satisfied, the flow proceeds to step 424. If the relevant rules are not satisfied, the flow proceeds to step 414. In step 424, a message is sent to the PBM/payer 104 that a PA is recommended for approval for the requested patient and/or medication. For instance, a signal is sent to PBM/payer 104 indicating that the medication is recommended for approval.

In step 426, the medication is noted as approved in PBM/payer 104. In step 428, a message is sent to the EHR and/or pharmacy that the PA is not needed, is approved, or some other similar message. For instance, PBM/payer 104 may return a message indicating that the PA is not needed or provide a message to EHR 108 indicating that the PA is approved. In step 430, the medication is released to the pharmacy. In step 432, a pharmacy claim is processed successfully based on the approved PA. It is noted that in various embodiments, transaction interception and approval system 106 may not need perform additional interventions and/or access additional question sets for automatically approving a PA, as described herein. If any of the relevant rules are not satisfied, the PA initiation request may be forwarded (e.g., unaltered) to PBM/payer 104 for processing in another fashion (e.g., manually).

FIG. 5 shows a flowchart 500 for a second illustrative implementation of a transaction interception, in accordance with an example embodiment. Further structural and operational examples will be apparent to persons skilled in the relevant art(s) based on the following descriptions. Flowchart 500 is described as follows with respect to system 100 of FIG. 1 and/or system 300 of FIG. 3. One or more of the steps of flowchart 500 may be performed by PBM/payer 104, transaction interception and approval system 106, EHR 108, and/or clinical data source 110. In various examples, one or more of the steps of flowchart 500 may be performed in a similar fashion as the steps of flowchart 400. For the sake of brevity, certain details with respect to flowchart 400 are not repeated in the following description of flowchart 500.

Flowchart 500 begins at step 502. In step 502, a new prescription (NewRx) is sent to pharmacy 102. In some examples, the NewRx is initiated at EHR 108. In other examples, the PA may be initiated at another entity not expressly shown.

In step 504, pharmacy 102 receives the NewRx. In step 506, transaction interception and approval system 106 receives the NewRx. In some implementations, transaction interception and approval system 106 receives the NewRx from pharmacy 102 (or another entity, such as PBM/payer 104). In other examples, transaction interception and approval system 106 may intercept the NewRx after initiation (e.g., by intercepting the communication between EHR 108 and pharmacy 102).

In step 508, an intelligent filtering model determines a probability of success of adjudicating the NewRx. For instance, transaction interception and approval system 106 may apply intelligent transaction filtering as described herein (e.g., by applying model 310) to determine if the NewRx (which includes a potential need for the PA to be associated with the NewRx) should be processed by transaction interception and approval system 106 (e.g., for automatic adjudication of the NewRx). In examples, the potential need for the PA to be associated with the NewRx may arise where a NewRx does not include a flag indicating that a PA is needed (in which case the NewRx can be flagged with such an indication). If the probability is above a threshold, the flow proceeds to step 510. If the probability is not above the threshold, the flow proceeds to step 514.

In step 510, authorization rules for a payer and/or medication associated with the request are obtained. For example, transaction interception and approval system 106 may access a set of rules for determining whether the NewRx should be processed by transaction interception and approval system 106. For instance, the rules can include a combination of medication (e.g., NDC or equivalent code) and plan information to determine if there is a potential for automatically approving the NewRx and/or computable, codified rules that evaluate clinical data against the NewRx requirements. In step 512, a determination is made whether rules exist for intervention (e.g., rules for the NDC/plan combination). If rules exist for intervention (e.g., rule(s) for the NDC/plan combination), the flow proceeds to step 516. If rules do not exist for intervention, the flow proceeds to step 514.

At step 514, the NewRx is forwarded (e.g., unaltered) to a pharmacy, which may process the NewRx in another fashion (e.g., manually). For instance, in step 514, a pharmacy may run a test claim to see if a PA is needed and follows an ordinary/usual process.

At step 516, transaction interception and approval system applies an intelligent filtering model (e.g., model 310) an additional time (e.g., by analyzing more information associated with the NewRx) to determine a probability of success of automatic adjudication. If the probability is above a threshold, the flow proceeds to step 518. If the probability is not above the threshold, the flow proceeds to step 514.

In step 518, clinical data is retrieved from a clinical data source (e.g., patient data source 534, which is an example implementation of clinical data source 110) based on rule requirements. For instance, if a rule exists for an NDC/plan combination and/or the probability of adjudication is above a threshold, clinical data may be obtained from EHR 108 and/or patient data source 534 (or any other source which contains patient medical information and/or history, accessed via any suitable means or system).

In step 520, an outcome is determined (e.g., an adjudication of the NewRx, which may comprise a recommended adjudication), and the intelligent filtering model is updated with the outcome. For instance, model 310 may be updated with the outcome to improve its accuracy.

In step 522, a determination is made whether approval rules are satisfied from available data. For instance, transaction interception and approval system 106 may evaluate the clinical data against computable, codified rules to determine if relevant rules are satisfied to approve medication (e.g., approve the NewRx). If relevant rules are satisfied, the flow proceeds to step 524. If the relevant rules are not satisfied, the flow proceeds to step 514. In step 524, a message is sent to the PBM/payer 104 that a PA is recommended for approval for the requested patient and/or medication. For instance, a signal is sent to PBM/payer 104 indicating that the medication is recommended for approval. In step 526, the medication is noted as approved in PBM/payer 104.

In step 528, the prescription is sent to a pharmacy (e.g., pharmacy 102). In step 530, the pharmacy runs a test claim to determine if a PA is needed. In step 532, the pharmacy accesses test claim notes (e.g., by communicating with PBM/payer 104) that indicate that a PA is not needed, and the medication is dispensed.

In embodiments, transaction interception and approval system 106 may not need additional interventions and/or access additional question sets for automatically approving (or recommending for approval) a PA or NewRx if all the relevant rules are satisfied, as described herein. If all relevant rules are satisfied, when the pharmacy sends a claim for the medication, PBM/payer 104 may be configured to approve (or recommend for approval) the prescription without any further prior authorization in such an example. If any of the relevant rules are not satisfied, the NewRx may be forwarded (e.g., unaltered) to pharmacy 102 for processing in another fashion (e.g., manually).

In embodiments, transaction interception and approval system 106 may use a routing system as a source of prescriptions to evaluate for prior authorizations. In embodiments, routing, electronic prior authorizations, new prescription information, prescription change information, formulary information and/or directory information may be used as sources of data for training the model as described herein.

In embodiments, transaction interception and approval system 106 may allow current standards (e.g., the National Council for Prescription Drug Programs (NCPDP) Electronic Prior Authorization standard) to continue to function without change while creating an alternative, automated approach that requires minimal or no changes to the EHR and minimal changes to PBM processing systems. In embodiments, implementation of the disclosed techniques may be carried out so as to be “invisible” to the PBM and/or EHR.

Accordingly, disclosed techniques allow for a reduction in the administrative burden of processing prior authorizations for both providers and payers (in addition to other technical benefits as described herein).

While disclosed techniques relate to automatic adjudication of electronic prior authorizations, implementations are not so limited. In other embodiments, the disclosed techniques may be used for adjudication of prior authorization of medical benefits needs (e.g., medical services). In some embodiments, the disclosed techniques may be triggered by an electronic event, such as an order for a medical service, to gather information and process other rules, such as identifying a gap in care or to retrieve more clinical data to support the needs of the pharmacy.

### III. Further Example Embodiments and Advantages

As described, systems and devices embodying the techniques herein may be configured and enabled in various ways to perform their respective functions. In some example embodiments, one or more of the operations of the flowcharts and/or flow diagrams described herein may not be performed. Moreover, operations in addition to or in lieu of the operations of the flowcharts and/or flow diagrams described herein may be performed. Further, in some example embodiments, one or more of the operations of the flowcharts and/or flow diagrams described herein may be performed out of order, in an alternate sequence, or partially (or completely) concurrently with each other or with other operations.

Embodiments and techniques, including methods, described herein may be performed in various ways such as, but not limited to, being implemented by hardware, or hardware combined with one or both of software and firmware. As described herein, systems, devices, components, etc., of the embodiments that are configured to perform functions and/or operations are also contemplated as performing such functions and/or operations.

### IV. Example Processing Device Implementations

Embodiments described herein may be implemented in hardware, or hardware combined with software and/or firmware. For example, embodiments described herein may be implemented as computer program code/instructions configured to be executed in one or more processors and stored in a computer readable storage medium. Alternatively, embodiments described herein may be implemented as hardware logic/electrical circuitry.

As noted herein, the embodiments described, including but not limited to, system 100 in FIG. 1, along with any components and/or subcomponents thereof, as well any operations and portions of flowcharts/flow diagrams described herein and/or further examples described herein, may be implemented in hardware, or hardware with any combination of software and/or firmware, including being implemented as computer program code configured to be executed in one or more processors and stored in a computer readable storage medium, or being implemented as hardware logic/electrical circuitry, such as being implemented together in a system-on-chip (SoC), a field programmable gate array (FPGA), an application specific integrated circuit (ASIC), a trusted platform module (TPM), and/or the like. A SoC may include an integrated circuit chip that includes one or more of a processor (e.g., a microcontroller, microprocessor, digital signal processor (DSP), etc.), memory, one or more communication interfaces, and/or further circuits and/or embedded firmware to perform its functions. In embodiments with respect to the example computer implementations in this Section, main memory, memory cards and memory sticks, memory devices, and/or the like may include and or implement the described techniques and embodiments.

Embodiments described herein may be implemented in one or more computing devices similar to a mobile system and/or a computing device in stationary or mobile computer embodiments, including one or more features of mobile systems and/or computing devices described herein, as well as alternative features. The descriptions of computing devices provided herein are provided for purposes of illustration, and are not intended to be limiting. Embodiments may be implemented in further types of computer systems, as would be known to persons skilled in the relevant art(s).

The embodiments described herein, including devices, systems, methods/processes, and/or apparatuses, may be implemented in or using processing devices, communication systems, servers, and/or, computers, such as a processing device 600 shown in FIG. 6. It should be noted that processing device 600 may represent mobile devices, communication devices/systems, entertainment systems/devices, processing devices, and/or traditional computers in one or more embodiments. For example, a resource generation system as described herein, and any of the sub-systems and/or components respectively contained therein and/or associated therewith, along with further embodiments thereof, may be implemented in or using one or more processing devices 600 and/or similar computing devices.

Processing device 600 can be any commercially available and well known communication device, processing device, and/or computer capable of performing the functions described herein. For example, processing device 600 may be a mobile computing device such as a handheld computer (e.g., a personal digital assistant (PDA)), a laptop computer, a tablet computer (such as an Apple iPad™), a hybrid device, a notebook computer (e.g., a Google Chromebook™ by Google LLC), a netbook, a mobile phone (e.g., a cell phone, a smart phone such as an Apple® iPhone® by Apple Inc., a phone implementing the Google® Android™ operating system, etc.), a wearable computing device (e.g., a head-mounted augmented reality and/or virtual reality device including smart glasses such as Google® Glass™, Oculus Rift® of Facebook Technologies, LLC, etc.), or other type of mobile computing device. Processing device 600 may alternatively be a stationary computing device such as a desktop computer, a personal computer (PC), a stationary server device, a minicomputer, a mainframe, a supercomputer, etc.

Processing device 600 includes one or more processors (also called central processing units, or CPUs), such as a processor 606. Processor 606 is connected to a communication infrastructure 602, such as a communication bus. In some embodiments, processor 606 can simultaneously operate multiple computing threads, and in some embodiments, processor 606 may comprise one or more processors.

Processing device 600 also includes a primary or main memory 608, such as random access memory (RAM). Main memory 608 has stored therein control logic 624 (computer software), and data.

Processing device 600 also includes one or more secondary storage devices 610. Secondary storage devices 610 include, for example, a hard disk drive 612 and/or a removable storage device or drive 614, as well as other types of storage devices, such as memory cards and memory sticks. For instance, processing device 600 may include an industry standard interface, such a universal serial bus (USB) interface for interfacing with devices such as a memory stick. Removable storage drive 614 can include any receptacle of or otherwise coupled to processing device 600 for receiving information from or otherwise being in communication with removable storage unit 616.

Removable storage drive 614 interacts with a removable storage unit 616. Removable storage unit 616 includes a computer useable or readable storage medium 618 having stored therein computer software 626 (control logic) and/or data. Removable storage unit 616 can include any suitable removable memory device type, including an SD (Secure Digital) card, a Subscriber Identity Module (SIM) card, which is well known in GSM (Global System for Mobile Communications) communication systems, a flash drive, an optical storage disk, and/or other removable physical memory device type for storage of computer data. Removable storage drive 614 reads from and/or writes to removable storage unit 616 in a well-known manner.

Processing device 600 also includes input/output/display devices 604, such as touchscreens, LED and LCD displays, monitors, keyboards, pointing devices, etc.

Processing device 600 further includes a communication or network interface 620. Communication interface 620 enables processing device 600 to communicate with remote devices. For example, communication interface 620 allows processing device 600 to communicate over communication networks or mediums 622 (representing a form of a computer useable or readable medium), such as LANs, WANs, the Internet, etc. Network interface 620 may interface with remote sites or networks via wired or wireless connections.

Control logic 628 may be transmitted to and from processing device 600 via the communication medium 622.

Any apparatus or manufacture comprising a computer useable or readable medium having control logic (software) stored therein is referred to herein as a computer program product or program storage device. This includes, but is not limited to, processing device 600, main memory 608, secondary storage devices 610, and removable storage unit 616. Such computer program products, having control logic stored therein that, when executed by one or more data processing devices, cause such data processing devices to operate as described herein, represent embodiments.

Techniques, including methods, and embodiments described herein may be implemented by hardware (digital and/or analog) or a combination of hardware with one or both of software and/or firmware. Techniques described herein may be implemented by one or more components. Embodiments may comprise computer program products comprising logic (e.g., in the form of program code or software as well as firmware) stored on any computer useable medium, which may be integrated in or separate from other components. Such program code, when executed by one or more processor circuits, causes a device to operate as described herein. Devices in which embodiments may be implemented may include storage, such as storage drives, memory devices, and further types of physical hardware computer-readable storage media. Examples of such computer-readable storage media include, a hard disk, a removable magnetic disk, a removable optical disk, flash memory cards, digital video disks, random access memories (RAMs), read only memories (ROM), and other types of physical hardware storage media. In greater detail, examples of such computer-readable storage media include, but are not limited to, a hard disk associated with a hard disk drive, a removable magnetic disk, a removable optical disk (e.g., CDROMs, DVDs, etc.), tapes, magnetic storage devices, MEMS (micro-electromechanical systems) storage, nanotechnology-based storage devices, flash memory cards, digital video discs, RAM devices, ROM devices, and further types of physical hardware storage media. Such computer-readable storage media may, for example, store computer program logic, e.g., program modules, comprising computer executable instructions that, when executed by one or more processor circuits, provide and/or maintain one or more aspects of functionality described herein with reference to the figures, as well as any and all components, capabilities, and functions therein and/or further embodiments described herein.

Such computer-readable storage media are distinguished from and non-overlapping with communication media and modulated data signals (i.e., do not include communication media or modulated data signals). Communication media embodies computer-readable instructions, data structures, program modules or other data in a modulated data signal such as a carrier wave. The term “modulated data signal” means a signal that has one or more of its characteristics set or changed in such a manner as to encode information in the signal. By way of example, and not limitation, communication media includes wireless media such as acoustic, RF, infrared and other wireless media, as well as wired media and signals transmitted over wired media. Embodiments are also directed to such communication media.

The techniques and embodiments described herein may be implemented as, or in, various types of circuits, devices, apparatuses, and systems. For instance, embodiments may be included, without limitation, in processing devices (e.g., illustrated in FIG. 6) such as computers and servers, as well as communication systems such as switches, routers, gateways, and/or the like, communication devices such as smart phones, home electronics, gaming consoles, entertainment devices/systems, etc. A device, as defined herein, is a machine or manufacture as defined by 35 U.S.C. § 101. That is, as used herein, the term “device” refers to a machine or other tangible, manufactured object and excludes software and signals. Devices may include digital circuits, analog circuits, or a combination thereof. Devices may include one or more processor circuits (e.g., central processing units (CPUs), processor 606 of FIG. 6), microprocessors, digital signal processors (DSPs), and further types of physical hardware processor circuits) and/or may be implemented with any semiconductor technology in a semiconductor material, including one or more of a Bipolar Junction Transistor (BJT), a heterojunction bipolar transistor (HBT), a metal oxide field effect transistor (MOSFET) device, a metal semiconductor field effect transistor (MESFET) or other transconductor or transistor technology device. Such devices may use the same or alternative configurations other than the configuration illustrated in embodiments presented herein.

### V. Additional Example Embodiments

A method for filtering transactions over a network is disclosed herein. The method is performed by a device that includes at least one processor and a memory that stores program instructions that, when executed by the at least one processor, perform a method comprising: retrieving, over the network, a transaction request; accessing a model configured to predict a likelihood associated with automatic adjudications of transactions; based at least on the transaction request and the model, determining a likelihood that the transaction request can be adjudicated automatically; if the likelihood that the transaction request can be adjudicated automatically is above a threshold, performing one of: generating an indication that at least a portion of the transaction request can be adjudicated automatically, or generating an automatic adjudication indicating whether the transaction request should be approved; and in response to generating the indication or the automatic adjudication, forwarding the transaction request and one of the indication or the automatic adjudication over the network to an endpoint associated with the transaction request.

In one implementation of the foregoing method, the model comprises one or more of a machine learning model or a set of predefined rules.

In another implementation of the foregoing method, the transaction request comprises a request for a prior authorization for a medical good or service.

In another implementation of the foregoing method, the generating the automatic adjudication of the transaction request comprises recommending approval of the transaction request based at least on an analysis of one or more of clinical, demographic, or payer data associated with a patient.

In another implementation of the foregoing method, the generating the automatic adjudication of the transaction request comprises recommending approval of the transaction request based at least on an analysis of one or more of: capabilities of a source technology platform, or an interoperability characteristic of the source technology platform.

In another implementation of the foregoing method, the retrieving the transaction request comprises intercepting a new order for a medical good or service.

In another implementation of the foregoing method, the retrieving the transaction request comprises intercepting an initiation of a prior authorization.

In another implementation of the foregoing method, the method further comprises: if the likelihood that the transaction request can be adjudicated automatically is not above the threshold, forwarding the transaction request unaltered to the endpoint.

In another implementation of the foregoing method, the retrieving the transaction request comprises one of: intercepting the transaction request over the network before the endpoint receives the transaction request, or retrieving the transaction request from the endpoint.

In another implementation of the foregoing method, the portion of the transaction request comprises a question of a question set associated with the transaction request, and the generating the indication comprises retrieving data to answer the question.

A system for filtering transactions over a network is disclosed herein. The system includes: a processor; and a memory device that stores program code structured to cause the processor to: retrieve, over the network, a transaction request; access a model configured to predict a likelihood associated with automatic adjudications of transactions; based at least on the transaction request and the model, determine a likelihood that the transaction request can be adjudicated automatically; if the likelihood that the transaction request can be adjudicated automatically is above a threshold, perform one of: generating an indication that at least a portion of the transaction request can be adjudicated automatically, or generating an automatic adjudication indicating whether the transaction request should be approved; and in response to generating the indication or the automatic adjudication, forward the transaction request and one of the indication or the automatic adjudication over the network to an endpoint associated with the transaction request.

In one implementation of the foregoing system, the model comprises one or more of a machine learning model or a set of predefined rules.

In another implementation of the foregoing system, the transaction request comprises a request for a prior authorization for a medical good or service.

In another implementation of the foregoing system, the program code is structured to cause the processor to generate the automatic adjudication of the transaction request by: recommending approval of the transaction request based at least on an analysis of one or more of clinical, demographic, or payer data associated with a patient.

In another implementation of the foregoing system, the program code is structured to cause the processor to generate the automatic adjudication of the transaction request by: recommending approval of the transaction request based at least on an analysis of one or more of: capabilities of a source technology platform, or an interoperability characteristic of the source technology platform.

In another implementation of the foregoing system, the program code is structured to cause the processor to retrieve the transaction request by: intercepting a new order for a medical good or service.

In another implementation of the foregoing system, the program code is structured to cause the processor to retrieve the transaction request by: intercepting an initiation of a prior authorization.

In another implementation of the foregoing system, the program code is structured to cause the processor to: if the likelihood that the transaction request can be adjudicated automatically is not above the threshold, forward the transaction request unaltered to the endpoint.

In another implementation of the foregoing system, the program code is structured to cause the processor to retrieve the transaction request by one of: intercepting the transaction request over the network before the endpoint receives the transaction request, or retrieving the transaction request from the endpoint.

In another implementation of the foregoing system, the portion of the transaction request comprises a question of a question set associated with the transaction request, and the generating the indication comprises retrieving data to answer the question.

A system for generating a machine-learning model to identify prior authorization requests that meet a threshold for further processing by a transaction approval system is disclosed herein. The system includes: a processor; and a memory device that stores program code structured to cause the processor to: retrieve prior authorization training data corresponding to tracked prior authorizations; generate a plurality of vectors based at least on one or more of the prior authorization training data, a prior authorization history, a prescription history, or a data source providing clinical data; generate combined prior authorization vectors based at least on the plurality of vectors; and generate a recommendation model using a supervised machine-learning algorithm based at least on the combined prior authorization vectors, the recommendation model configured to identify a likelihood that a retrieval of clinical data will satisfy a set of rules configured for the least one of a payer or medication identified in a transaction request.

In one implementation of the foregoing system, the plurality of vectors comprises one or more of: prior authorization result vectors corresponding to outcomes based on the prior authorization history; provider vectors corresponding to a requesting provider based on the prior authorization history; data source vectors corresponding to the data source providing one or more of clinical data or administrative data; medical goods or services vectors corresponding to a medical good or service requested based on the prior authorization history; payer vectors corresponding to a payer based on the prior authorization history; questionset vectors corresponding to a questionset based on the prior authorization history; diagnosis code vectors corresponding to a diagnosis code set based on the prior authorization history and a medical goods or services history; or clinical data vectors corresponding to clinical data of a patient; and wherein the vector combiner is configured to generate the combined prior authorization vectors based on the one or more of the result, provider, data source, medical goods or services, payer, questionset, diagnosis code vectors, or clinical data vectors.

### VI. Conclusion

While various embodiments have been described above, it should be understood that they have been presented by way of example only, and not limitation. It will be apparent to persons skilled in the relevant art that various changes in form and detail can be made therein without departing from the spirit and scope of the embodiments. Thus, the breadth and scope of the embodiments should not be limited by any of the above-described exemplary embodiments, but should be defined only in accordance with the following claims and their equivalents.

What is claimed is:

1. A method for filtering transactions over a network performed by a device that includes at least one processor and a memory that stores program instructions that, when executed by the at least one processor, perform a method comprising:
retrieving, over the network, a transaction request;
accessing a model configured to predict a likelihood associated with automatic adjudications of transactions;
based at least on the transaction request and the model, determining a likelihood that the transaction request can be adjudicated automatically;
if the likelihood that the transaction request can be adjudicated automatically is above a threshold, performing one of:
generating an indication that at least a portion of the transaction request can be adjudicated automatically, or
generating an automatic adjudication indicating whether the transaction request should be approved; and
in response to generating the indication or the automatic adjudication, forwarding the transaction request and one of the indication or the automatic adjudication over the network to an endpoint associated with the transaction request.

2. The method of claim 1, wherein the model comprises one or more of a machine learning model or a set of predefined rules.

3. The method of claim 1, wherein the transaction request comprises a request for a prior authorization for a medical good or service.

4. The method of claim 1, wherein the generating the automatic adjudication of the transaction request comprises recommending approval of the transaction request based at least on an analysis of one or more of clinical, demographic, or payer data associated with a patient.

5. The method of claim 1, wherein the generating the automatic adjudication of the transaction request comprises recommending approval of the transaction request based at least on an analysis of one or more of:
capabilities of a source technology platform, or
an interoperability characteristic of the source technology platform.

6. The method of claim 1, wherein the retrieving the transaction request comprises intercepting a new order for a medical good or service.

7. The method of claim 1, wherein the retrieving the transaction request comprises intercepting an initiation of a prior authorization.

8. The method of claim 1, further comprising:
if the likelihood that the transaction request can be adjudicated automatically is not above the threshold, forwarding the transaction request unaltered to the endpoint.

9. The method of claim 1, wherein the retrieving the transaction request comprises one of:
intercepting the transaction request over the network before the endpoint receives the transaction request, or
retrieving the transaction request from the endpoint.

10. The method of claim 1, wherein the portion of the transaction request comprises a question of a question set associated with the transaction request, and
wherein the generating the indication comprises retrieving data to answer the question.

11. A system for filtering transactions over a network, the system comprising:
a processor; and
a memory device that stores program code structured to cause the processor to:
retrieve, over the network, a transaction request;
access a model configured to predict a likelihood associated with automatic adjudications of transactions;
based at least on the transaction request and the model, determine a likelihood that the transaction request can be adjudicated automatically;
if the likelihood that the transaction request can be adjudicated automatically is above a threshold, perform one of:
generating an indication that at least a portion of the transaction request can be adjudicated automatically, or
generating an automatic adjudication indicating whether the transaction request should be approved; and
in response to generating the indication or the automatic adjudication, forward the transaction request and one of the indication or the automatic adjudication over the network to an endpoint associated with the transaction request.

12. The system of claim 11, wherein the model comprises one or more of a machine learning model or a set of predefined rules.

13. The system of claim 11, wherein the transaction request comprises a request for a prior authorization for a medical good or service.

14. The system of claim 11, wherein the program code is structured to cause the processor to generate the automatic adjudication of the transaction request by:
recommending approval of the transaction request based at least on an analysis of one or more of clinical, demographic, or payer data associated with a patient.

15. The system of claim 11, wherein the program code is structured to cause the processor to generate the automatic adjudication of the transaction request by:
recommending approval of the transaction request based at least on an analysis of one or more of:
capabilities of a source technology platform, or
an interoperability characteristic of the source technology platform.

16. The system of claim 11, wherein the program code is structured to cause the processor to retrieve the transaction request by:
intercepting a new order for a medical good or service.

17. The system of claim 11, wherein the program code is structured to cause the processor to retrieve the transaction request by:
intercepting an initiation of a prior authorization.

18. The system of claim 11, wherein the program code is structured to cause the processor to:
if the likelihood that the transaction request can be adjudicated automatically is not above the threshold, forward the transaction request unaltered to the endpoint.

19. The system of claim 11, wherein the program code is structured to cause the processor to retrieve the transaction request by one of:
intercepting the transaction request over the network before the endpoint receives the transaction request, or
retrieving the transaction request from the endpoint.

20. The system of claim 11, wherein the portion of the transaction request comprises a question of a question set associated with the transaction request, and
wherein the generating the indication comprises retrieving data to answer the question.

21. A system for generating a machine-learning model to identify prior authorization requests that meet a threshold for further processing by a transaction approval system, the system comprising:
a processor; and
a memory device that stores program code structured to cause the processor to:
retrieve prior authorization training data corresponding to tracked prior authorizations;
generate a plurality of vectors based at least on one or more of the prior authorization training data, a prior authorization history, a prescription history, or a data source providing clinical data;
generate combined prior authorization vectors based at least on the plurality of vectors; and
generate a recommendation model using a supervised machine-learning algorithm based at least on the combined prior authorization vectors, the recommendation model configured to identify a likelihood that a retrieval of clinical data will satisfy a set of rules configured for the least one of a payer or medication identified in a transaction request.

22. The system of claim 21, wherein the plurality of vectors comprises one or more of:
prior authorization result vectors corresponding to outcomes based on the prior authorization history;
provider vectors corresponding to a requesting provider based on the prior authorization history;
data source vectors corresponding to the data source providing one or more of clinical data or administrative data;
medical goods or services vectors corresponding to a medical good or service requested based on the prior authorization history;
payer vectors corresponding to a payer based on the prior authorization history;
questionset vectors corresponding to a questionset based on the prior authorization history;
diagnosis code vectors corresponding to a diagnosis code set based on the prior authorization history and a medical goods or services history; or
clinical data vectors corresponding to clinical data of a patient; and
wherein the vector combiner is configured to generate the combined prior authorization vectors based on the one or more of the result, provider, data source, medical goods or services, payer, questionset, diagnosis code vectors, or clinical data vectors.
